(19) United States
(12) Patent Application Publication
(10) Pub. No.: US 2008/0221597 A1
(43) Pub. Date: Sep. 11, 2008

(54) Methods and devices for intragastrointestinal fixation

(76) Inventors: Jeffrey M. Wallace, Charlestown, RI (US); Peter J. Lukin, Norfolk, MA (US); Donald Coelho, Bellinghm, MA (US); Gregory Amante, Holliston, MA (US)

Correspondence Address: DAVOL INC.;(Anthony Colesanti, Esq.), 100 Sockanossett Crossroad, Cranston, RI, 02920, US

(21) Appl. No.: 11/269,393

(22) Filed: Nov. 8, 2005

Related U.S. Application Data
(60) Provisional application No. 60/725,778, filed on Oct. 12, 2005.

(51) Int. Cl. A61B 17/08

(52) U.S. Cl. 606/157

(57) ABSTRACT

A device for fixating a gastrointestinal device may include a band of material that may be wrapped around the gastrointestinal tract, for example, by wrapping the band around the outside of the stomach or wrapping the band in such a way that it is integrated into and/or with the stomach tissue. In this illustrative embodiment, the band restricts the stomach such that the stomach lumen is constricted and/or the stomach is restricted from expanding beyond what the band will allow.

This application claims priority to U.S. Provisional Patent Application Ser. No. 60/725,778, filed Oct. 12, 2005, which is hereby incorporated by reference in its entirety.

### FIELD OF INVENTION

This present invention relates to methods and devices for intragastrointestinal fixation.

### BACKGROUND

Obesity, as defined by a body mass index (BMI) of 30 kg/m² or more, is a rapidly growing problem, currently affecting more than 30% of adults in the United States. Morbid obesity, as defined by a body mass index of 40 kg/m² or more or a BMI of 35 kg/m² or more in the presence of co-morbidities is also prevalent, affecting 3.1% of men and 6.7% of women. Obesity is commonly associated with many serious medical disorders including heart disease, diabetes, hypertension, hyperlipidemia, hypercholesterolemia, osteoarthritis and sleep apnea. In addition, approximately 300,000 adults in the U.S. die each year due to obesity-related causes.

The primary treatment objective for obese patients is weight reduction, which can improve co-morbid conditions and also reduces risk factors for disease. Even moderate weight loss (5%-10% of initial weight) produces health benefits and has been associated with marked reductions in the risk for the medical disorders listed above. While non-operative and pharmacologic weight loss therapies have met with only limited success, surgical intervention for morbid obesity, most frequently gastric bypass, is becoming increasingly common. However, the decision to undergo gastric bypass is a difficult one. Patients who choose to undergo gastric bypass are making a serious commitment to permanent life-style changes and are at risk for developing metabolic/nutritional complications resulting from the long-term malabsorptive effects of gastric bypass and food intake restriction. Long-term complications of gastric bypass including anemia secondary to iron or B₁₂ deficiency, mineral deficiencies (hypokalemia and hypomagnesia) and bone disease associated with secondary hyperparathyroidism are not uncommon. These conditions can be serious thereby necessitating lifelong medical follow-up to monitor for such events.

Although various procedures exist for the surgical treatment of morbid obesity, the Roux-en-Y gastric bypass (RYGB) has been identified as the gold standard for morbidly obese patients when non-invasive interventions have failed. The RYGB procedure entails the creation of a small gastric pouch to which the distal jejunum is attached via creation of an anastomosis referred to as a gastrojejunostomy (GJ). The procedure excludes more than 95% of the stomach, all of the duodenum and the proximal jejunum from digestive continuity. Weight loss is thought to result from reduced intake volume due to the small gastric pouch and limited GJ diameter, as well as from malabsorption due to the bypass of the proximal jejunum. The procedure is associated with a mean of 65-75% excess weight loss with 1% mortality and 10% morbidity.

Despite the favorable safety and effectiveness profile of the RYGB procedure, technical complications and inadequate weight loss may occur. Serious complications are not uncommon after open bariatric procedures. Adhesion formation may contribute to small bowel obstructions, which may require an additional operation for the patient. Incisional hernias are another complication associated with abdominal surgical procedures and have been shown to occur at a much higher rate after open gastric bypass surgery than after laparoscopic bypass surgery.

The significant morbidity associated with traditional weight loss surgery emphasizes the importance of the development of minimally invasive interventions that will result in patient weight loss, which may improve co-morbid conditions and also reduce risk factors for disease. Additionally, a minimally invasive or intragastrointestinal approach will minimize or eliminate many of the risks associated with open and laparoscopic procedures.

### BRIEF DESCRIPTION OF THE DRAWINGS

Certain embodiments of the invention are described with reference to the accompanying drawings, which, for illustrative purposes, are not necessarily drawn to scale.

FIG. 1 illustrates an embodiment of the invention wherein a band of material may create a stricture point or narrowing that may provide fixation for an intragastrointestinal device or prosthesis.

FIG. 2 illustrates an embodiment of an intragastrointestinal device or prosthesis with a flanged portion that may assist in fixation. An optional distal portion is also shown.

FIG. 3 illustrates an embodiment of an intragastrointestinal device or prosthesis with an extended portion that may assist in fixation. An optional distal portion is also shown.

### DETAILED DESCRIPTION

Presented here are illustrative embodiments of an invention for fixating an intragastrointestinal device, including but not limited to an intragastrointestinal prosthesis, in place within, but not limited to, at least a portion of a gastrointestinal lumen. The gastrointestinal tract may include, but is not limited to, the stomach, esophagus, intestines, colon, rectum, anus, and other areas continuous with the gastrointestinal lumen. In one preferred embodiment of the present invention a band of material is wrapped around the gastrointestinal tract, for example by wrapping the band around the outside of the stomach or wrapping the band in such a way that it is integrated into and/or with the stomach tissue. In this configuration, the band restricts the stomach such that the stomach lumen is constricted and/or the stomach is restricted from expanding beyond what the band will allow. Certain embodiments of the present invention may include the use of a band of material to constrict the lumen of the gastrointestinal tract, creating a narrowing or a stricture point within the said lumen. This portion of the gastrointestinal tract lumen where the narrowing or constriction occurs may be defined as the stricture point. This stricture point or narrowing may be created at any point on the stomach. As an example, the band may be placed around the proximal portion of the stomach near the fundus. In another embodiment, a band may be wrapped around the proximal portion of the stomach near the esophagus. By positioning the band near the esophagus, the volume of the portion of the stomach between the stricture point and the gastroesophageal junction may be specified. In preferred embodiments of the present invention, the volume of the proximal portion of the stomach between the stricture point and the gastroesophageal junction is within the range of about 10-30 ml.

A band according to the present invention is made of a biocompatible material, which may in certain embodiments include non-absorbable collagen fibers. Such a band may be wrapped around the stomach and once the band has been implanted, the stomach tissue may integrate the collagen fibers into its structural matrix. As a result, the collagen fibers may work with the stomach tissue to create a longer lasting constriction or stricture point or narrowing due to the fact that the collagen fibers may reinforce stomach tissue in the shape and size determined when the band was implanted. Such an embodiment may be beneficial, as the stricture point or narrowing within the gastrointestinal tract lumen may be reinforced by the collagen fibers in the band, while the band may resist migration or tearing through the tissue into the lumen due to the fact that the collagen fibers may have integrated into the tissues structural matrix.

Certain embodiments of the present invention may include the use of a band, whether made out of non-absorbable collagen fibers or some other material, to constrict and/or restrict the gastrointestinal tract, which may create a narrowing of said lumen. The dimension of the lumen may be controlled in the embodied system by controlling the amount of constriction the band applies. As a result, the present embodiment may be designed such that the dimensions of the lumen at a stricture point or narrowing is kept large enough to prevent the stricture point or narrowing from significantly impeding the flow of swallowed food, particles or fluid as it passes through the gastrointestinal tract. While the stricture point or narrowing in the embodied system may be a wide enough dimension to not significantly inhibit food or fluid passage, the dimension may be selected so it is small enough to prevent larger objects, including but not limited to intragastrointestinal devices or prosthesis, or intragastrointestinal artificial sphincters from passing through the stricture point. Objects placed within the gastrointestinal tract lumen that may restrict the amount of food the subject intakes may be defined as a restrictive device element. Objects/devices placed within the lumen that restrict the absorption of components of the swallowed food or fluid into the body, including but not limited to, the absorption of nutrients, minerals, and fluids into the body, may be defined as a malabsorptive element. Therefore, the stricture point or narrowing created by the band of material may prevent an intragastrointestinal device or prosthesis that is placed in the proximal portion from moving into the distal portion of the lumen. According to this configuration the stricture point or narrowing can act as a seat or fixation point for the intragastrointestinal device or prosthesis to sit and not migrate within the gastrointestinal tract lumen. It should also be noted that in some embodiments of the invention, the stricture point or narrowing created by the band may prevent an intragastrointestinal device or prosthesis from moving from the distal portion into the proximal portion of the lumen. In such embodiments, the stricture point or narrowing may act as a seat for the intragastrointestinal device or prosthesis to sit or be fixated on the distal portion of the gastrointestinal tract.

In embodiments of the invention where an intragastrointestinal device or prosthesis that incorporates a restrictive device element as at least one of its functional tasks is seated at the stricture point or narrowing, the stricture point or narrowing can be placed in a manner that the effective stomach volume can be reduced allowing the subject to reach satiety quickly and reduce food intake. In such embodiments, a determined volume of the proximal portion of the stomach (a portion close to the esophagus) may be achieved such that as food enters the proximal portion of the stomach from the esophagus, the intragastrointestinal device or prosthesis seated at the stricture point or narrowing may regulate or restrict the flow of food from the proximal portion of the stomach into the distal portion of the stomach. In this embodiment or similar embodiments, the proximal portion of the stomach may fill quickly and potentially cause the subject to reach satiety and stop eating. This may result in a decrease in food intake in comparison to the food intake of a normal subject that has no intragastrointestinal devices or stricture points in their stomach. It also should be noted that in some embodiments of the present invention, if the dimension of the lumen at the stricture point or narrowing is designed to be large enough to not significantly impede swallowed food or fluid as it passes through the gastrointestinal tract, the stricture point or narrowing by itself may not act as a restrictive device element. Therefore, in embodiments where an intragastrointestinal device or prosthesis that incorporates a restrictive device element, is seated at the stricture point or narrowing, by removing said intragastrointestinal device or prosthesis, either through means including but not limited to removing said device or prosthesis from the seat or removing said device or prosthesis from the gastrointestinal tract, the effect or effects of the restrictive device element may be minimized or removed, and swallowed food or fluid may flow through the stomach without being significantly impeded or restricted. It should also be noted that embodiments of the present invention may also include a stricture point or narrowing that may act as a seat for an intragastrointestinal device or prosthesis, where at least a portion of an intragastrointestinal device or prosthesis may reside outside of the gastrointestinal tract lumen.

In certain embodiments of the invention, a stricture point or narrowing may act as a seat or a fixation point for a device or prosthesis that is not an intragastrointestinal device or prosthesis. As such, the stricture point or narrowing may act as a seat or fixation point for non-intragastrointestinal devices or prosthesis, or it may act as a seat or fixation point for intragastrointestinal devices or prosthesis, or it may act as a seat or fixation point for devices and/or prosthesis that are comprised of elements that are a combination of intragastrointestinal and non-intragastrointestinal.

Certain embodiments of the present invention include methods and/or techniques for implanting or placing a band around at least a portion of the gastrointestinal tract to create a stricture point or narrowing. The implantation or placing of band may be done through methods included, but not limited to open surgery, laparoscopic surgery, minimally invasive surgery or flexible endoscopy. Such embodiments may include the use of an oro-gastric tube or some other instrument that is of a known diameter, which may be introduced into the gastrointestinal tract lumen via a body orifice. With the oro-gastric tube or some other tool of a known diameter within the lumen, the band can be placed around at least a portion of the gastrointestinal tract at a particular position. In this embodiment and other similar embodiments, the band can be attached around at least a portion of the stomach that contains the oro-gastric tube or known diameter tool, and the constriction that may create a stricture point or narrowing of known dimension, based on the diameter of the oro-gastric tube or known diameter tool.

Embodiments of the present invention include methods and/or techniques for implanting the band around at least a portion of the gastrointestinal tract with the band cut to a designated length prior to or during implantation. By selecting the proper length of the band, when the band is fixated around at least a portion of the gastrointestinal tract, the resulting dimension of the stricture point or narrowing may be of a predetermined value based on the length of the band. Additionally, the calculations used to determine the proper length of the band in certain embodiments can also compensate for factors including, but not limited to wall thickness, band overlap, tissue elasticity, band elasticity, and compliance of the a particular gastrointestinal tract tissue.

Certain embodiments of the invention may allow the implantation of the band that will constrict the lumen of the gastrointestinal tract to a determined diameter. Certain embodiments of the invention may allow the implantation of the band that will constrict the lumen to reduce the compliance of gastroesophageal junction. This reduction of compliance in the lower esophageal sphincter may restrict the flow of stomach contents into the esophagus and/or prevent aspiration of fluids.

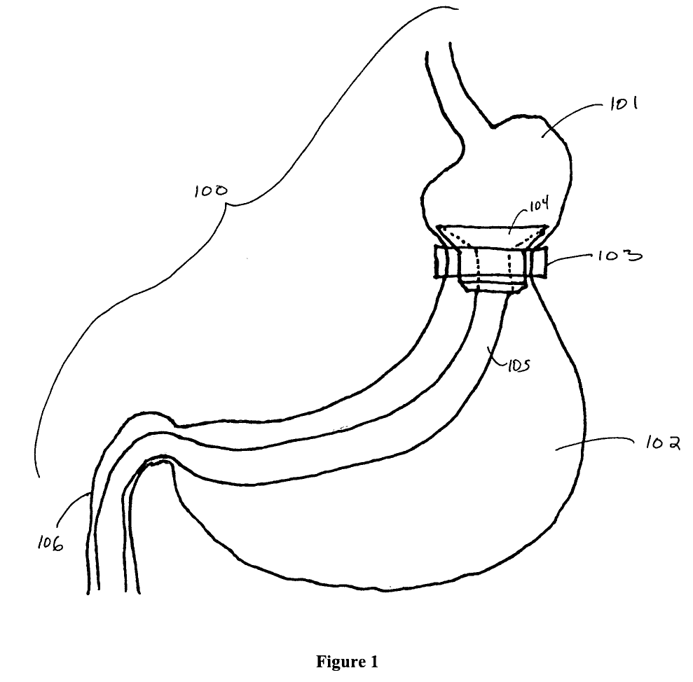

In FIG. 1, an embodiment of the invention is illustrated as it relates to the stomach (100), although other embodiments in other portions of the gastrointestinal tract are similarly possible. A material or band (103) is wrapped around a portion of the stomach. This band of material (103) may be made of, but is not limited to, a non-absorbable collagen fiber band which may integrate into the stomach wall structure. The band of material (103) may be composed of some other material that may be biocompatible. The band of material (103) may constrict or restrict the size of the stomach lumen. This restriction may create a reduced lumen or stoma within the stomach that separates the upper or proximal portion of the stomach (101) and the lower or distal portion of the stomach (102). The stoma or stricture point or narrowing in the preferred embodiment may act as a seat or fixation point for an intragastrointestinal or partially intragastrointestinal object include, but not limited to prosthesis, restrictive device elements, and malabsorptive elements.

In the preferred embodiment, an intrgastric prosthesis device (illustrated as 104 and 105) may be held in place or seated by the stricture point or narrowing caused by the band of material (103). The embodied device or prosthesis (104 & 105) may preferably have a proximal portion (104) with a flanged or enlarged portion where said portion's dimensions may be greater than an inner dimension of the stricture point or narrowing caused by the band of material (103). This may prevent the proximal portion of the prosthesis or device (104) from moving from the upper portion of the stomach (101) into the lower portion (102). Optionally, the preferred embodiment of a device or prosthesis (104 & 105) may also include a distal portion (105) that extends into the lower portion of the stomach (102) and it may extend into the pylorus and/or intestines. The distal portion (105) in the illustrated embodiment may act as a conduit and/or a malabsorptive device element, preventing the stomach and/or intestines from absorbing components from the swallowed food or fluid. Additionally, in the preferred embodiment, the distal portion (105) may provide a counter force that may prevent the proximal portion (104) and/or the embodied device from disengaging or moving away from the stricture point or narrowing caused by the band of material (103). Such embodiments may be implemented through means including, but not limited to, utilizing contact force or frictional force or resistive force which may be integrated along at least a portion of the distal portion. This may be implemented where the portion of the distal portion makes contact with tissue. This may also be implemented by fixating a portion of the distal portion to the tissue which may reside distally from the stricture point or narrowing. It should be noted that in the preferred embodiment, the dimensions or diameter of the stricture point or narrowing may be large enough such that it does not act as a restrictive element when considered by itself in the stomach. As such, if an embodied device or prosthesis is removed from the stricture point or narrowing, the stricture point or narrowing may not act as a restrictive device element, and if the embodied device or prosthesis did act as a restrictive device element, that effect may be minimized or removed.

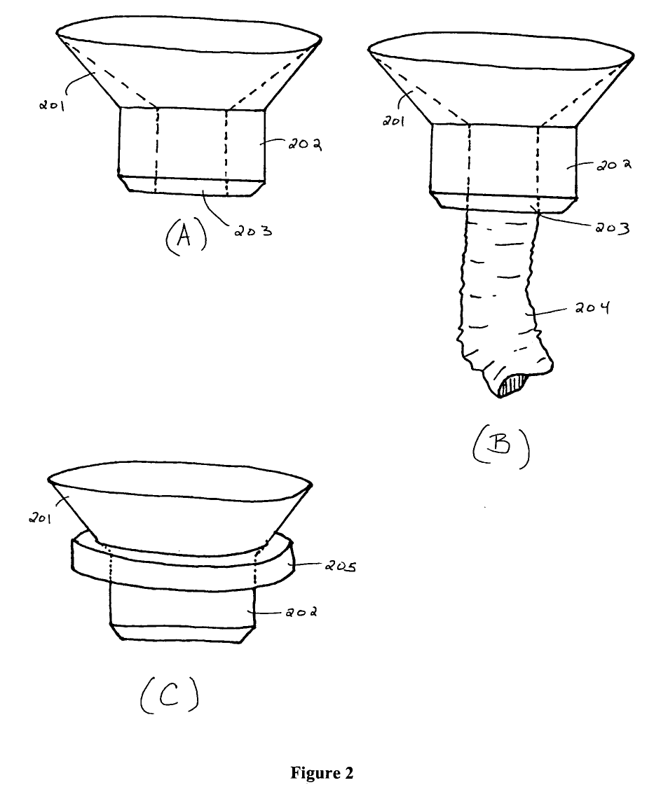

FIG. 2 illustrates potential embodiments of an intragastrointestinal device or prosthesis. Such an embodiment may be seated at a stricture point or narrowing where the lumen (203) of the embodiment may act as a restrictive device element. The illustrated embodiment incorporates a flanged portion (201) which may have a greater dimension than the body portion (202). As illustrated in FIG. 2A, the embodied intragastrointestinal device or prosthesis may incorporate an optional restrictive device element. Optionally, as illustrated in FIG. 2B, the embodied device may include a distal portion (204). Such a distal portion (204) may be a continuation of the body portion's lumen (203). Such a distal portion (204) may incorporate a malabsorptive device element. In the preferred embodiment, the greatest dimension of the flanged portion (201) may be larger than a inner dimension of the stricture point or narrowing. This embodied aspect is illustrated in FIG. 2C. In said embodiments, the body portion (202) has a dimension which may be less than a inner dimension of a stricture point or narrowing (illustrated in FIG. 2C as element 205). Additionally, in embodiments of the present invention illustrated in FIG. 2C, the flanged portion (201) may have a dimension which may be greater than a inner dimension of a stricture point or narrowing (205). In these or similar embodiments, the body portion may move through the stricture point or narrowing from the upper portion of the stomach to the lower portion (or understandably from the lower portion to the upper portion of the stomach). However, the dimension of the flanged portion (201) may be larger than the inner dimension of the stricture point or narrowing (205), and as a result, the embodied intragastrointestinal device or prosthesis may not be able to pass through a stricture point or narrowing. In such embodiments, the device may be fixated or seated on the stricture point or narrowing, allowing the intragastrointestinal device or prosthesis to remain in place.

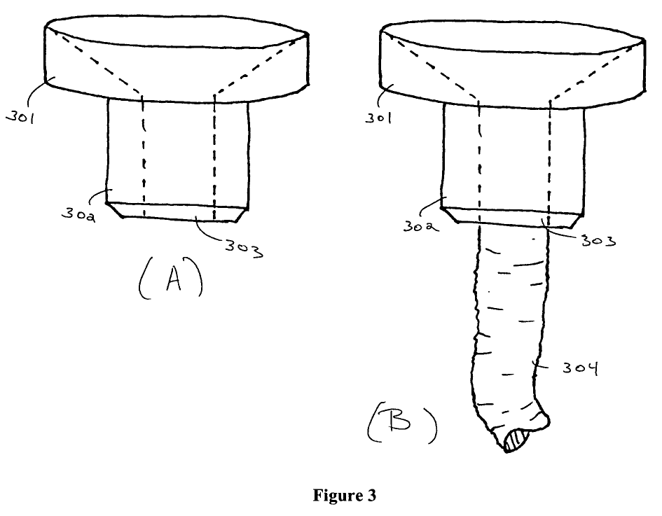

Various other embodiments of the inventions are also possible. One such embodiment is illustrated in FIG. 3. This embodiment is similar to the embodiment shown in FIG. 2 and shares many of its aspects. Such an embodiment may be seated at a stricture point or narrowing where the lumen (303) of the embodiment may act as a restrictive device element. In the illustrated embodiment, the embodied intragastrointestinal device or prosthesis may incorporate a flanged or ring element (301) which may have a greater dimension than the body portion (302) or a greater dimension than a stricture point or narrowing (which is not illustrated in FIG. 3, but may be similar to embodiments shown in FIG. 2C). As illustrated in FIG. 3A, the embodiment of the intragastrointestinal device or prosthesis may optionally comprise a restrictive device element. Optionally, as illustrated in FIG. 3B, the distal portion (304) of the device may be incorporated into the embodiment. Such an incorporation distal portion (304) may be a continuation of the body portion's lumen (303). Such a distal portion (304) may optionally incorporate a malabsorptive device element.

When certain embodiments of an intragastrointestinal device or prosthesis becomes fixated or seated on a stricture point or narrowing, the forces applied on the tissue may be such that tissue damage could occur. Such tissue damage may include, but is not limited to, compression damage or injury. As a result, certain embodiments of the present invention may contain protective elements that will potentially help guard against tissue damage. One embodiment of the present invention would include the use of a sponge-like substance or coating on the surface of an embodied intragastrointestinal device or prosthesis where the embodied device comes into contact with the tissue. Additional embodiments may include the use of a gel-like substance or coating between the embodied device and tissue. In such embodiments and other using similar methods to accomplish the same, the protective element may cushion the tissue and potentially reduce the amount of tissue damage that may occur.

It is, of course, understood that modification of the present invention, in its various aspects, will be apparent to those skilled in the art. Additional method and device embodiments are possible, their specific features depending upon the particular application. For example, multiple flanged portions may be used to aid fixation. Additionally, certain embodiment may be applicable to other portions of the gastrointestinal tract not named herein. Further, certain embodiments may be applicable to other organ system in addition to the gastrointestinal tract.

What is claimed is:

1. A method for fixating a device or prosthesis comprising:
fixating in place at least a portion of the device or prosthesis within at least a portion of the gastrointestinal tract lumen;
using at least a portion of a band of material to constrict or restrict the lumen size of said portion of the gastrointestinal tract lumen;
the constriction and/or restriction of said portion of the gastrointestinal tract lumen does not significantly impede the flow of food and/or fluid as it passes through said portion of the gastrointestinal tract lumen;
the constriction and/or restriction of said portion of the gastrointestinal tract lumen will prevent at least a portion of the device or prosthesis from passing through said portion of the gastrointestinal tract lumen; and
the constriction and/or restriction of said portion of the gastrointestinal tract lumen will fixate in place at least a portion of the device or prosthesis within at least a portion of said gastrointestinal tract lumen.

2. A method as in claim 1, wherein the band of material is wrapped around or integrated into at least a portion of the gastrointestinal tract.

3. A method as in claim 2, wherein at least a portion of the band of material is bicompatible and will integrate into or become part of the structural matrix of at least a portion of the tissue that makes up said portion of the gastrointestinal tract.

4. A method as in claim 3, wherein the integration into or becoming part of the structural matrix of said portion of the gastrointestinal tract tissue creates or reinforces the constriction and/or restriction of said portion of the gastrointestinal lumen.

5. A method as in claim 3, wherein at least a portion of the band of material comprises non-absorbable collagen fibers.

6. A method as in claim 3, wherein at least one of the functional aspects of the device or prosthesis is selected from the group consisting of: a restrictive device element, a malabsorptive device element, and a combination of a restrictive device element and a malabsorptive device element.

7. A method as in claim 2, wherein the implantation of at least a portion of the band of material is accomplished by at least one method or a combination of methods from the group consisting of: an open surgical procedure, a laproscopic surgical procedure, a flexible endoscopy procedure.

8. A method as in claim 2, wherein the dimension of at least a portion of the band of material is determined by at least one method or a combination of methods selected from the group consisting of: using at least a portion of the band of material where the dimension is determined prior to the procedure, using at least a portion of the band with the dimension altered to a certain dimension during the procedure, inserting an instrument of know dimension into said portion of the gastrointestinal lumen and subsequently wrapping a portion of the band of material around said portion of the gastrointestinal lumen including the instrument of known dimension.

9. A method as in claim 3, wherein the said portion of the gastrointestinal tract comprises at least a portion of the stomach lumen.

10. A method as in claim 9, wherein the constriction or restriction of a least a portion of the stomach is used at least part of a medical procedure selected from the group consisting of: a bariatric procedure, a gastrointestinal procedure, a general surgery procedure, a procedure related to gastric reflux disease.

11. A fixation device comprising:
at least a portion of a band of material composed at least in part of collagen fibers that will integrate into or become part of at least a portion of the gastrointestinal tract tissue;
at least a portion of a band of material will cause a constriction or restriction of at least a portion of the gastrointestinal lumen;
the constriction or restriction of at least a portion of the gastrointestinal tract lumen caused by the fixation device will not significantly impede the flow of swallowed food and/or fluid as it passed through said portion of the gastrointestinal tract lumen; and
the constriction or restriction of at least a portion of the gastrointestinal tract lumen caused by the fixation device will cause an intragastrointestinal device or prosthesis to fixate in place within said portion of the gastrointestinal tract lumen.

12. A fixation device, as in claim 11, wherein a portion of the band of material will integrate into or become part of the structural matrix of at least a portion of the gastrointestinal tract tissue creating or reinforcing the constriction or restriction of said portion of the gastrointestinal tract lumen.

13. A fixation device as in claim 11, wherein the intragastrointestinal device that is fixated in place by the fixation device includes at least one or a combination of functional aspects selected from the group consisting of: a restrictive device element, a malabsorptive device element, a restrictive and malabsorptive device element.

14. A fixation device as in claim 11, wherein the fixation device is used as part of at least a portion of a medical procedure selected from the group consisting of; a bariatric procedure, a gastrointestinal procedure, a general surgery procedure, a procedure related to gastric reflux disease.

15. An intragastrointestinal device comprising:
a flanged portion where the greatest dimension of the flanged portion is greater than the greatest dimension of the body portion;
the greatest dimension of the flanged portion is greater that the greatest internal dimension of a stricture point within at least a portion of the gastrointestinal tract lumen; and
the intragastrointestinal device may engage or seat on the stricture point such that the intragastrointestinal device is fixated in place.

16. An intragastrointestinal device as in claim 15, wherein at least a portion of the intragastrointestinal device has a dimension smaller than the inner dimension than the inner dimension of the stricture point such that at least a portion of the intragastrointestinal device resides within the stricture point and/or in the opposite side of the lumen of the stricture point from where the flanged portion resides.

17. An intragastrointestinal device as in claim 15, wherein the intragastrointestinal device incorporates at least one of or a combination of functional aspects selected from the group consisting of: a restrictive device element, a malabsorptive device elements, a restrictive and a malabsorptive device element.

18. An intragastrointestinal device as in claim 15, wherein the intragastrointestinal device is used as part of at least a portion of a medical procedure selected from a group consisting of: a bariatric procedure, a gastrointestinal procedure, a general surgery procedure, a procedure related to gastric reflux disease.
